(19) United States
(12) Patent Application Publication
(10) Pub. No.: US 2025/0288762 A1
(43) Pub. Date: Sep. 18, 2025

(54) Non-Invasive Ventilation Masks Having Purged Dual Seals

(71) Applicant: Creare LLC, Hanover, NH (US)

(72) Inventors: Daniel Micka, Lebanon, NH (US); Darin Knaus, Lyme, NH (US); Mark Bagley, Grafton, NH (US); Jeffrey Cox, Lebanon, NH (US)

(21) Appl. No.: 18/860,265

(22) PCT Filed: Jun. 21, 2023
(86) PCT No.: PCT/US2023/025848

§ 371 Date: Oct. 25, 2024

Related U.S. Application Data
(60) Provisional application No. 63/354,999, filed on Jun. 23, 2022.

(51) Int. Cl. A61M 16/00; A61M 16/06; A61M 16/10

(52) U.S. Cl. CPC A61M 16/0012; A61M 16/0616; A61M 16/105; A61M 2205/7509

(57) ABSTRACT

Ventilation masks that have breathing regions and inner and outer wearer-engaging seals and interseal regions between the inner and outer seals that are purged with either a vacuum or a positive pressure so as to prevent contaminated breathing gases from leaking into the environments surrounding the masks. In vacuum-purged embodiments, the pressure in the interseal region is lower than the pressures in both the breathing region and the surrounding environment. In some vacuum-purged embodiments, the vacuum is created using a vacuum ejector integrated into the mask. In pressure-purged embodiments, the pressure in the interseal region is greater than the pressures in both the breathing region and the surrounding environment. In some pressure-purged embodiments, the positive pressure is provided directly from a gas source, with a mask-integrated pressure reducer located between the gas source and the breathing region to reduce the pressure in the breathing region. Corresponding methods are also disclosed.

### RELATED APPLICATION DATA

This application claims the benefit of priority of U.S. Provisional Patent Application Ser. No. 63/354,999, filed Jun. 23, 2022, and titled “NON-INVASIVE VENTILATION MASK WITH PURGED DUAL SEAL”, which is incorporated herein by reference in its entirety.

### FIELD

The present disclosure generally relates to the field of ventilation masks. In particular, the present disclosure is directed to non-invasive ventilation masks having purged dual seals.

### BACKGROUND

Many patients with respiratory diseases, such as COVID-19, require positive pressure ventilation treatment. Effective and safe ventilation treatment of patients with highly infectious diseases poses several challenges. The best treatment for each patient varies based on the unique circumstances and the disease specifics, but currently available ventilation treatment options are suboptimal.

Invasive Ventilation. Invasive ventilation involves inserting an endotracheal tube into the oropharynx and into the trachea of a patient (intubation). A mechanical ventilator performs the majority (or in some cases all) of the breathing functions for a critically ill patient. This can provide immediate improvement in vital functions of the most critically ill patients but can cause significant side effects and some poor long-term outcomes. Existing alternatives to invasive ventilation can increase the risk of disease transmission through leakage of infectious droplets. However, due to the potential complications, it is not in the best interest of patients to be placed on invasive instead of noninvasive ventilation solely to reduce the risk of disease transmission. Additionally, there is a limited supply of mechanical ventilators, personnel trained to manage patients on a ventilator, and hospital rooms (e.g., intensive care unit (ICU) beds) set up to handle intubated patients.

Non-Invasive Positive Pressure Ventilation (NPPV). Non-invasive ventilation (NIV) is a less invasive and more conservative form of respiratory therapy compared to invasive ventilation. The elevated pressure airflow is supplied to the patient via a mask (or helmet interface). This type of support is associated with fewer complications than intubation and can lead to shorter hospitalization and ICU stays. A typical type of NPPV uses a Constant Positive Airway Pressure (CPAP) machine or other ventilator to provide a continuous flow of air delivered at a constant pressure. The pressurized air is typically delivered via a face mask that covers the mouth and nose. NPPV leads to improved alveolar recruitment, a particularly important benefit in patients infected with COVID-19.

Risk of Disease Transmission from Mask Leakage. Unfortunately, in patients with highly contagious respiratory conditions such as COVID-19, NPPV can lead to aerosolization of infectious respiratory droplets, which increases the risk of infectious transmission to individuals not wearing appropriate personal protective equipment. To reduce this risk, the recommended practice is to use a non-vented mask with a viral filter on the mask exhaust port. An example of a flow circuit for this type of non-vented mask 10 is shown in FIG. 1. As seen in FIG. 1, a CPAP machine 14 provides pressurized breathing air 12 to the conventional non-vented mask 10, and a viral filter 18 filters the exit gas 22 from the non-vented mask. This type of non-vented mask circuit greatly reduces the risk of virus spreading to the environment 26 via the exit gas 22. However, due to imperfect sealing against the patient's face 30, unfiltered, contaminated gas 34 will leak past the mask seal 10S into the surrounding environment 26 because the internal mask pressure, P1, is higher than the ambient pressure, P0. Therefore, despite the medical benefits, the use of NIV with infectious disease patients is controversial and is often discouraged due to the risk of disease transmission.

### SUMMARY

In one implementation, the present disclosure is directed to a ventilation mask for ventilating a wearer of the ventilation mask via at least one of a nose and a mouth of the wearer when the ventilation mask is surrounded by an ambient environment having an ambient pressure, P₀. The ventilation mask includes a breathing region designed and configured to receive the at least one of the nose and the mouth and contains breathing gases at a breathing pressure, P₁, when the ventilation mask is in use, wherein the breathing region has a periphery; an inner seal that defines the periphery of the breathing region and is designed and configured to engage the wearer during use of the ventilation mask so as to create a first gas seal between the wearer and the ventilation mask so as to seal the breathing region; an outer seal spaced from the inner seal so as to define an interseal region between the inner seal and the outer seal, the outer seal designed and configured to engage the wearer during use of the ventilation mask so as to create a second gas seal between the wearer and the ventilation mask so as to seal the interseal region; a gas-handling component that has either: a vacuum-purge-pressure configuration by which, during use, the gas-handling component receives a fresh breathing gas from a gas source at the breathing pressure P₁ and includes a vacuum generator that is in fluid communication with, separately, each of the breathing region and the interseal region, wherein the vacuum generator is designed and configured to, during use, exhaust the breathing gases from the breathing region and use the breathing gases at the breathing pressure P₁ to create a vacuum purge pressure, P_{V}, in the interseal region, wherein P_{V}<P₀<P₁; or a positive-purge-pressure configuration by which, during use, the gas-handling component receives the fresh breathing gas from the gas source at a positive purge pressure, P_{P}, that is higher than the breathing pressure P₁, provide the fresh breathing gas to the interseal region at the positive purge pressure P_{P}, and provide the fresh breathing gas received at the positive purge pressure P_{P} to the breathing region at the breathing pressure P₁, wherein P_{P}>P₁>P₀.

In another implementation, the present disclosure is directed to a method of ventilating, at a breathing pressure, P₁, higher than an ambient pressure, P₀, a mask wearer having a nose and a mouth via a ventilation mask having a breathing region containing at least one of the nose and the mouth and that has a purged dual seal engaged with the mask wearer and that seals the breathing region, the method comprising either: providing a vacuum purge pressure, P_{V}, to the purged dual seal so that P_{V}<P₀<P₁, wherein the providing of the vacuum purge pressure P_{V} includes: receiving fresh breathing gas at the breathing pressure P₁; and generating the vacuum purge pressure P_{V} from breathing gases exhausted from the breathing region; or providing a positive purge pressure, P_{P}, to the purged dual seal so that P_{P}>P₁>P₀, wherein the providing of the pressure purge pressure P_{P} includes: receiving a fresh breathing gas at a positive purge pressure P_{P}; providing the received fresh breathing gas to the purged dual seal at the positive purge pressure P_{P}; and providing the received fresh breathing gas to the breathing region at the breathing pressure P₁ by reducing pressure of the received fresh breathing gas from the positive purge pressure P_{P} to the breathing pressure P₁.

### BRIEF DESCRIPTION OF THE DRAWINGS

For the purpose of illustration, the accompanying drawings show aspects of one or more embodiments made in accordance with the present disclosure. However, it should be understood that the scope of this disclosure is/are not limited to the precise arrangements and instrumentalities shown in the drawings, wherein:

FIG. 1 is a diagram illustrating a gas-flow circuit for a conventional non-vented mask, showing how leakage of contaminated gas can occur between the mask seal and a patient's face;

FIG. 2 is a diagram illustrating a gas-flow circuit for a vacuum-purged ventilation mask made in accordance with aspects of the present disclosure;

FIG. 3 is a diagram illustrating a gas-flow circuit for a pressure-purged ventilation mask made in accordance with aspects of the present disclosure;

FIG. 4A is cross-sectional view of an example vacuum-purged (VP) ventilation mask of the present disclosure and having integrated VP components, showing details of the VP ventilation mask and gas flows;

FIG. 4B is a perspective view of the VP ventilation mask of FIG. 4A, showing exterior features of the VP ventilation mask;

FIG. 4C is a perspective cutaway view of the VP ventilation mask of FIG. 4A, showing features of the VP ventilation mask from a perspective different from FIG. 4A;

FIG. 5A is a perspective view of another example VP ventilation mask that is similar to the VP ventilation mask of FIGS. 4A through 4C but that further includes an integrated viral filter;

FIG. 5B is a cross-sectional view of the VP ventilation mask of FIG. 5A, showing features internal to the viral filter;

FIG. 6A is an elevational partial view of an example purged dual seal (PDS) with vacuum collapse prevention features that can be used with a VP ventilation mask, such as the VP ventilation masks of FIGS. 2 and 4A through 4C, and 5A and 5B;

FIG. 6B is a perspective cutaway view of a portion of the PDS of FIG. 6A;

FIG. 7A is an elevational partial view of another example PDS with vacuum collapse prevention features that can be used with a VP ventilation mask, such as the VP ventilation masks of FIGS. 2, 4A through 4C, and 5A and 5B;

FIG. 7B is a perspective cutaway view of a portion of the PDS of FIG. 7A;

FIG. 8 is a composite of a perspective partial view of another example VP ventilation mask having a vacuum indicator and a pair of enlarged perspective cutaway partial views of the vacuum indicator depicting states of the vacuum indicator at differing states of vacuum in the interseal region of the VP ventilation mask;

FIG. 9 is a graph of motive flow rate versus vacuum leak rate at three motive flow pressures for a VP ventilation mask utilizing the vacuum ejector and viral filter of FIGS. 5A and 5B;

FIG. 10 is a graph of vacuum level versus vacuum leak rate at three motive flow pressures for the VP ventilation mask of FIG. 9;

FIG. 11 is a graph of vacuum level versus vacuum leak rate at three motive flow pressures with simulated wearer breathing for the VP ventilation mask of FIGS. 9 and 10;

FIG. 12A is a perspective cutaway view of an example pressure-purged (PP) ventilation mask of the present disclosure and having integrated PP components, showing details of the PP ventilation mask and gas flows; and

FIG. 12B is an enlarged perspective cutaway partial view of a portion of the PDS of the PP ventilation mask of FIG. 12A.

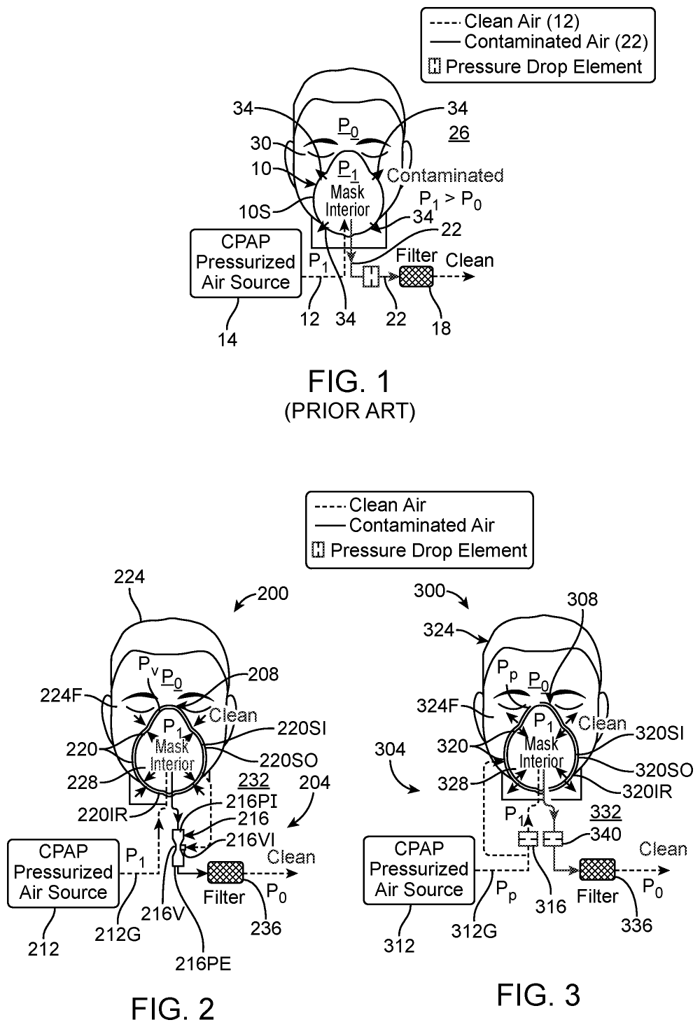

### DETAILED DESCRIPTION

### Overview

In some aspects, the present disclosure is directed to non-invasive positive pressure ventilation masks having purged dual seals (PDSs) that prevent leakage of infectious aerosols into the environment surrounding the ventilation masks, such as into patient-caregiver environments. In some embodiments, a PDS of this disclosure does not require any flow source other than the pressurized breathing air for the mask wearer. As described below in detail, a ventilation mask having a PDS of this disclosure has two seals that sealingly engage a wearer's body (typically the wearer's face), namely, an inner seal and an outer seal, and that define an interseal region therebetween. The inner seal is located between the interseal region and a breathing region of the ventilation mask at which the wearer of the ventilation mask breathes via their nose and/or mouth. The outer seal is located between the interseal region and the environment surrounding the ventilation mask. The interseal region is purged with a purge flow that is either a vacuum flow or a pressurized flow, depending on the particular design implemented.

A PDS ventilation mask of the present disclosure can be, for example, an oronasal mask (a/k/a “full face mask”) that covers the nose and mouth only, a total face mask that covers the nose, mouth, and eyes, or a helmet that covers the entire head and seals at the neck. That said, examples below are of the oronasal type, as these are the most common type in certain applications, such as health care applications. However, those skilled in the art will readily understand how to make the changes necessary to adapt the general principles of an oronasal PDS ventilation mask to another type of PDS ventilation mask, such as a total-face PDS ventilation mask or a PDS ventilation helmet. For the sake of convenience, the term “ventilation mask” as used herein and in the appended claims includes an oronasal ventilation mask, a full-face ventilation mask, and a ventilation helmet unless specifically indicated otherwise. In this connection, it is noted that the constructions of non-PDS full-face ventilation masks and non-PDS ventilation helmets are well known in the art such that persons of ordinary skill in the art will be able to integrate a PDS of the present disclosure into such constructions without undue experimentation.

Both vacuum-purge (VP) flow and pressure-purge (PP) flow in the interseal region of a PDS accomplish the same result, namely, preventing leakage of contaminated aerosols from the mask wearer to the ambient environment by providing a suitable leakage-prevention pressure gradients across the inner or outer seals. All of the contaminated air from the wearer's breathing is captured in a mask exhaust and may be passed, for example, through a viral filter that removes the contaminated aerosols. Such viral filters are widely used and effective devices. Embodiments of a PDS ventilation mask of the present disclosure have fault tolerant designs that do not rely on a perfect seal to prevent the release of contaminated air. This is particularly important, as minor air leakage, even with a good mask seal, is a near universal problem.

Beneficially, the purge flow of a PDS ventilation mask of the present disclosure is created from the pressurized breathing gas supplied to the patient. No additional external flow source, either pressurized or vacuum, is needed, and the pressurized breathing air can be supplied to the inlet of the ventilation mask using the same equipment that is used for conventional masks, for example a ventilator, a CPAP (Constant Positive Airway Pressure) machine, or a BiPAP (Bi-level Positive Airway Pressure) machine. Therefore, embodiments of a PDS ventilation mask made in accordance with the present disclosure can be used with the same tubing and connections as a standard non-vented CPAP/BiPAP circuit; no additional equipment, tubing or connections are needed in such embodiments. This makes embodiments of PDS ventilation masks of the present disclosure optimal for use in a critical care environment with existing NIV equipment and practices.

For VP-seal embodiments, the interseal region of the VP ventilation mask is purged by a vacuum pressure that is lower than the pressure in the ambient environment surrounding the VP ventilation mask and the wearer of the VP ventilation mask. This ensures that contaminated gases that the mask wearer exhales will not leak outward across the outer seal to the ambient environment. Any leakage across the outer seal will be inward toward the interseal region due to the pressure gradient. The vacuum purge flow is created by a vacuum ejector, which in some embodiments may be integrated into the VP ventilation mask. The motive flow for the vacuum ejector is the exhaust air flow, or “primary mask exhaust”, from the pressurized flow in the breathing region of the VP ventilation mask. This flow is at elevated pressure leaving the VP ventilation mask by way of a pressurized-gas source, such as a ventilator, CPAP machine, or BiPAP machine. The pressure drop for the exhaust from the breathing region then occurs across the motive-flow orifice of the vacuum ejector. Thus the vacuum ejector obviates the need for a secondary restrictive element in the exhaust circuit of a VP ventilation mask of the present disclosure.

The primary mask exhaust mixes with VP flow in the vacuum ejector, and the combined flow stream exits the vacuum ejector at a pressure that is slightly above the ambient pressure. The combined flow may optionally be routed, for example, to a viral filter that capture virus particles and/or other infectious particles that may be in the wearer's exhaled breath. In some embodiments, the VP ventilation mask may include one or more additional features, including but not limited to, a vacuum indicator that indicates that a suitable vacuum pressure is present in the interseal region, a mask-integrated filter, coaxial rotating elbow interface for coaxial flow, and anti-collapse features for the interseal region.

Example gas-flow circuits in a number of example VP ventilation masks and features are illustrated in FIGS. 2 and 4A through 8 and described below in detail. It is noted that while these examples each show a single vacuum ejector integrated into the corresponding VP ventilation mask, other embodiments may include more than one vacuum ejector and/or more than one motive-force nozzles and/or the vacuum ejector(s) can be deployed in a non-integrated manner. Regarding the use of multiple vacuum ejectors, when they all provide the same function of contributing to the vacuum-purge flow for the PDS, they may be collectively considered to function as a single vacuum ejector. As an example, multiple vacuum ejectors may handle drawing vacuum from multiple corresponding locations of the interseal region of a PDS. Regarding non-integrated vacuum ejectors, the wearer-engaging component of the VP ventilation mask comprising the PDS may, for example, have two separate connections for individually connecting, e.g., via separate gas conduits, a vacuum plenum for the PDS aboard the wearer-engaging component to a vacuum connector on the vacuum ejector and a primary exhaust outlet of the wearer-engaging component to the inlet of the vacuum ejector. As another example, one or more vacuum ejectors may be integrated into a wearer-engaging component of the VP ventilation mask that includes the PDS. Regardless of the location of the vacuum ejector(s) in a VP ventilation mask of the present disclosure, each vacuum ejector is a part of the VP ventilation mask.

In some embodiments in which the vacuum ejector is integrated into a VP ventilation mask, each vacuum ejector may be part of a gas-handling component that also includes coaxial passageways for the primary flow and the purge flow. In some embodiments, the gas-handling component may be part of an elbow assembly that is rotatably attached to the wearer-engaging component. In some embodiments, the coaxial passageways may be arranged so that the breathing region of the VP ventilation mask is supplied with pressurized gas via a central passageway and the PDS is provided with a purge vacuum via an annular passageway surrounding the central passageway. In some embodiments, the passageways may be reversed in function so that the purge vacuum is provided via the central passageway and the pressurized gas for breathing is provided via the surrounding annular passageway. In some embodiments, one or both of the central and annular passageways may be replaced by one or more passageways having alternative configurations. Those skilled in the art will be able to use the teachings herein of the underlying functionalities of the various passageways and components of VP ventilation masks of the present disclosure and ordinary knowledge in the art to design many variations without undue experimentation.

For PP-seal embodiments, the interseal region of a PP ventilation mask is maintained with a flow of clean, i.e., uncontaminated by exhaled breath of the mask wearer, gas that is at a pressure higher than the pressure in the breathing region of the VP ventilation mask. The relatively higher pressure in the interseal region ensures that no contaminated air will leak outward across the inner seal to the interseal region; all leakage across the inner seal will be inward to the breathing region due to the pressure gradient. The pressurized purge is obtained using the same source gas that is provided to the mask wearer as the breathing gas. The breathing gas supplied to the PP ventilation mask is supplied at a purge pressure that is higher than the breathing pressure. This higher-pressure flow is connected to the interseal region to provide the purge flow and then flows into the breathing region of the PP ventilation mask through a passive pressure-drop device that reduces the purge pressure to the desired breathing pressure. As a result, the pressure in the interseal region of the PP ventilation mask is higher than the pressure in the breathing region. The mask exhaust from the breathing region may optionally be routed, for example, to a viral filter that capture virus particles and/or other infectious particles that may be in the wearer's exhaled breath. In some embodiments, the PP ventilation mask may include one or more additional features, including but not limited to, a pressure indicator that indicates that the interseal region is at a suitable pressure higher than in the breathing region, a mask-integrated filter, coaxial rotating elbow interface for coaxial flow, and a passive pressure-drop device on the primary exhaust of the PP ventilation mask.

### Example VP Ventilation Masks, and VP Mask Systems and Methods

FIG. 2 shows an example VP mask system 200 of the present disclosure and the attendant gas-flow circuit 204. In this example, the VP mask system 200 includes a VP ventilation mask 208, one or more pressurized-gas sources 212, and a vacuum ejector 216. The VP ventilation mask 208 includes a PDS 220 that includes an inner seal 220SI and an outer seal 220SO that engage with the face 224F of a wearer 224 and that define between them an interseal region 220IR that is generally open to the wearer's face. In this context, “generally open” means that the interseal region 220IR is either directly open to the face 224F of the wearer 224 or that a gas-permeable barrier (not shown) is present but does not block passage of gas to the extent that the disclosed functionality of the PDS 220 is destroyed.

The inner seal 220SI and impermeable structure (not seen in FIG. 2) of the VP ventilation mask 208 define a breathing region 228 that, here, allows the wearer 224 to breath via her/his mouth and nose. When the VP ventilation mask 208 is properly engaged with the face 224F of the wearer 224, the breathing region 228 is completely fluidly isolated from the interseal region 220IR, except for any leakage that could potentially occur between the inner seal 220SI and the wearer's face. When the wearer 224 is wearing the VP ventilation mask 208, the parts of the VP ventilation mask not fluidly inward of the outer seal 220SO are exposed to the ambient environment 232. For the sake of describing the gas-flow circuit 204 of this example VP mask system 200, the following pressure variables are used: P₀ is the pressure in the ambient environment 232, P₁ is the pressure within the breathing region 228, and P_{V} is the pressure in the interseal region 220IR. All values provided for P₀, P₁, and P_{V} are values during proper functioning of the PV mask system 220.

The pressurized-gas source 212 may be any source(s) of one or more gases 212G (referred to hereinafter singly and collectively as “gas”), which may be oxygen, air, breathable medicament, water vapor, among others, and any logical combination thereof, that the wearer 224 can breathe during use of the VP ventilation mask 208. Each pressurized-gas source 212 may be any suitable source, such as an active source (e.g., pump) or a passive source (e.g., a pressurized tank and regulator), among others, and any suitable combination. Fundamentally, there are no limitations on each pressurized-gas source 212 other than that it supplies one or more suitable breathable gases 212G at the necessary pressure for a useful amount of time. Examples of pressurized-gas sources suitable for use as or in the pressurized-gas source(s) 212 include, but are not limited to a CPAP machine, a BiPAP machine, a ventilator machine, and a tank-and-regulator system, among others. Those skilled in the art will be familiar with pressurized-gas sources suitable for use as or in the pressurized-gas source(s) 212 such that further description is not necessary for them to practice the innovations disclosed herein to their fullest scope without undue experimentation.

A principle of operation of this example VP mask system 200 is to maintain the pressure P_{V} in the interseal region 220IR at a pressure that is lower than both the pressure P₁ within the breathing region 228 and the pressure P₀ in the ambient environment 232. In other words, in this example, P₁>P₀>P_{V}. To create this pressure scheme in this example, the pressurized-gas source 212 nominally provides its gas 212G at the pressure P₁ within the breathing region 228 of the VP ventilation mask 208. The term “nominally” is used to account for any loses that may occur, such as due to flow losses between the pressurized-gas source(s) 212 and the breathing region 228. Gas 212G within the breathing region 228 is exhausted from the breathing region and passes through the vacuum ejector 216.

In this example, the vacuum ejector 216 includes a venturi constriction 216V, containing a motive-force orifice (not shown), a primary inlet 216PI, a primary exhaust 216PE, and a vacuum inlet 216VI. The primary inlet 216PI is fluidly connected to the breathing region 228 of the VP ventilation mask 208, and the vacuum inlet 216VI is fluidly connected to the interseal region 220IR. During operation, the pressurized flow out of the breathing region 228, which is nominally at pressure P₁, flows through the venturi constriction 216V, thereby creating a low pressure that draws a vacuum, at pressure P_{V}, in the interseal region 220IR. As mentioned above, the pressure P_{V} within the interseal region 220IR is lower than both the pressure P₁ within the breathing region 228 and the pressure P₀ in the ambient environment 232. Consequently, if there is any leakage between either or both of the inner and outer seals 220SI and 220SO of the PDS, this vacuum effect will draw either any contaminated gas from the breathing region 228 or fresh air from the ambient environment 232, or both, into the interseal region 220IR and into the vacuum ejector 216, where it/they will be exhausted with the gas from the breathing region that entered the vacuum ejector via the primary inlet 216PI. Downstream of the venturi tube 216V, the vacuum ejector 216 exhausts the combined gas flows from the breathing region 228, for example, a viral filter 236 or other exhaust-handler, which may then exhaust the filtered gas into the ambient environment or other suitable location. It is noted that in other embodiments, the gas exhausted from the vacuum ejector 216 may be exhausted directly into the ambient environment in some applications.

Example Pressure-Purged Ventilation Masks and Pressure-Purged Mask System and Methods

FIG. 3 shows an example PP mask system 300 of the present disclosure and the attendant gas-flow circuit 304. In this example, the PP mask system 300 includes a PP ventilation mask 308, one or more pressurized-gas sources 312, and a pressure reducer 316. The PP ventilation mask 308 includes a PDS 320 that includes an inner seal 320SI and an outer seal 320SO that engage with the face 324F of a wearer 324 and that define between them an interseal region 320IR that is generally open to the wearer's face. In this context, “generally open” means that the interseal region 320IR is either directly open to the face 324F of the wearer 324 or that a gas-permeable barrier (not shown) is present but does not block passage of gas to the extent that the disclosed functionality of the PDS 320 is destroyed.

The inner seal 320SI and impermeable structure (not seen in FIG. 3) of the PP ventilation mask 308 define a breathing region 328 that, here, allows the wearer 324 to breath via her/his mouth and nose. When the PP ventilation mask 308 is properly engaged with the face 324F of the wearer 324, the breathing region 328 is completely fluidly isolated from the interseal region 320IR, except for any leakage that could potentially occur between the inner seal 320SI and the wearer's face. When the wearer 324 is wearing the PP ventilation mask 308, the parts of the PP ventilation mask not fluidly inward of the outer seal 320SO are exposed to the ambient environment 332. For the sake of describing the gas-flow circuit 304 of this example PP mask system 300, the following pressure variables are used: P₀ is the pressure in the ambient environment 332, P₁ is the pressure within the breathing region 328, and P_{P} is the pressure in the interseal region 320IR. All values provided for P₀, P₁, and P_{P} are values during proper functioning of the PP mask system 300.

The pressurized-gas source(s) 312 may be any source(s) of one or more gases 312G (referred to hereinafter singly and collectively as “gas”), which may be oxygen, air, breathable medicament, water vapor, among others, and any logical combination thereof, that the wearer 324 can breathe during use of the PP ventilation mask 308. Each pressurized-gas source 312 may be any suitable source, such as an active source (e.g., pump) or a passive source (e.g., a pressurized tank and regulator), among others, and any suitable combination. Fundamentally, there are no limitations on each pressurized-gas source 312 other than that it supplies one or more suitable breathable gases 312G at the necessary pressure for a useful amount of time. Examples of pressurized-gas sources suitable for use as or in the pressurized-gas source(s) 312 include, but are not limited to a CPAP machine, a BiPAP machine, a ventilator machine, and a tank-and-regulator system, among others. Those skilled in the art will be familiar with pressurized-gas sources suitable for use as or in the pressurized-gas source(s) 312 such that further description is not necessary for them to practice the innovations disclosed herein to their fullest scope without undue experimentation.

A principle of operation of this example PP mask system 300 is to maintain the pressure P_{P} in the interseal region 320IR at a pressure that is higher than both the pressure P₁ within the breathing region 328 and the pressure P₀ in the ambient environment 332. In other words, in this example, P_{P}>P₁>P₀. To create this pressure scheme in this example, the pressurized-gas source 312 nominally provides its gas 312G at the pressure P_{P} within the interseal region 320IR of the PDS 320. As above, the term “nominally” is used to account for any loses that may occur, such as due to flow losses between the pressurized-gas source(s) 312 and the interseal region 320IR.

As noted above, the pressure P_{P} in the interseal region 320IR is higher than the pressure P₁ in the breathing region 328. This is accomplished in this example by the pressurized-gas source 312 operating at a pressure, here, pressure P_{P}, higher than it would for a conventional ventilation mask that operates at the breathing pressure P₁. In this example, the two pressures P_{P} and P₁ are provided via a single pressurized-gas source 312 by the PP mask system providing a first portion of the gas 312G to the interseal region 320IR directly at pressure P_{P} and causing a pressure drop in a second portion of the gas from pressure P_{P} to pressure P₁, here via the pressure reducer 316, before providing the second portion of the gas to the breathing region 328. The pressure reducer 316 may be a suitable pressure-reducing element or device, such as a flow restriction or pressure regulator, among others. Fundamentally, there are no limitations on the type of pressure reducer used for the pressure reducer 316 other than it provides the necessary function.

With the pressure PP within the interseal region 320IP higher than both the pressures P1 and P0 in the breathing region 328 of the PP ventilation mask 308 and the ambient environment 332, respectively, any leakage that may occur between the inner seal 320SI and the face 324F of the wearer 324 will be of the clean gas 312G in the interseal region 320IR into the breathing region 328 due to the pressure gradient. Consequently, any contaminants in the exhaled breath of the wearer 324 with be contained in the breathing region 328. Likewise, any leakage that may occur between the outer seal 320SO and the face 324F of the wearer 324 will be of the clean gas 312G in the interseal region 320IR into the ambient environment 332.

In the embodiment shown, the gas within the breathing region 228, which may include, for example, infectious material exhaled by the wearer 324, is exhausted from the breathing region 328 and passes through a viral filter 336 or other exhaust-handler, which may then exhaust the filtered gas into the ambient environment 332 or other suitable location. It is noted that in other embodiments, the gas exhausted from the breathing region 328 may be exhausted directly into the ambient environment 332 in some applications. In this example, the PP mask system 300 includes an optional pressure reducer 340 located in the flow path of the exhausted gas between the breathing region 328 and the viral filter 336. Similar to the pressure reducer 316, the pressure reducer 340 may be any suitable type of pressure reducer element or device.

Example instantiations of the VP and PP ventilation masks 208 and 308 of FIGS. 2 and 3, respectively, are described in detail below.

### Detailed Examples

### VP Ventilation Masks

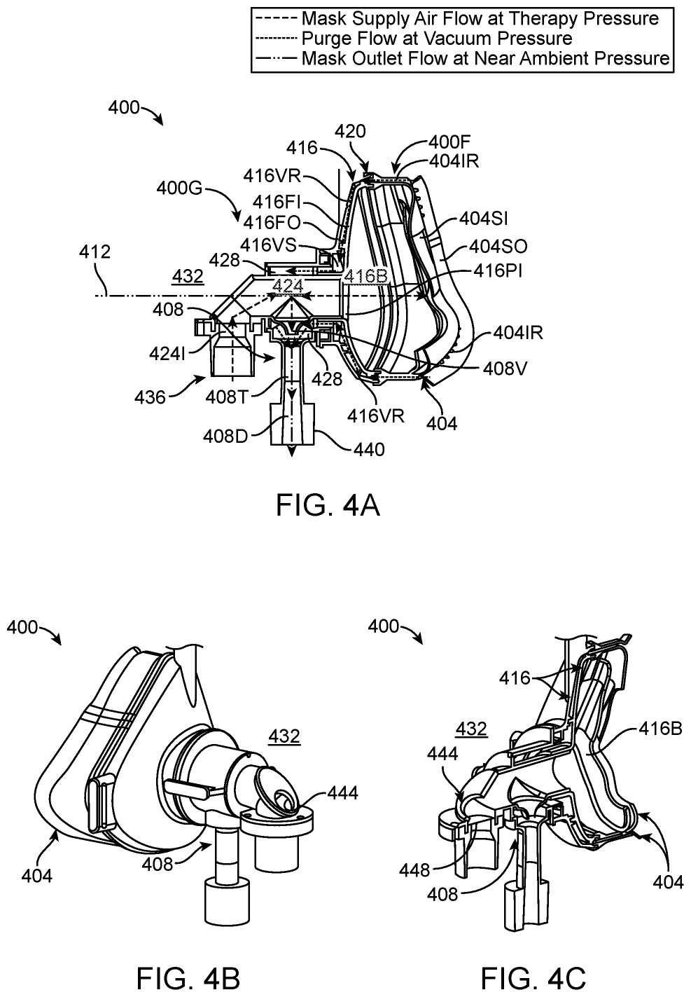

FIGS. 4A through 4C illustrate an example VP ventilation mask 400 that incorporates features and aspects of the present disclosure, including a PDS 404 and a vacuum ejector 408, which in this example is integrated into the VP ventilation mask so as to provide a unitary structure. At a high level, this instantiation of the VP ventilation mask 400 may be considered to have face-engaging component 400F and a gas-handling component 400G, which in this example is coupled to the face-engaging component so as to be pivotable relative to the face-engaging component about a pivot axis 412. This pivoting interface can be important for reducing the forces of connected tubing (not shown) on the VP ventilation mask 400 as the wearer (not shown), for example, a patient, moves.

In this embodiment, the face-engaging component 400F includes the PDS 404, which is secured to a body 416 that includes an inner frame 416FI and an outer frame 416FO that are spaced from one another so as to define a vacuum region 416VR therebetween. In this example, the body 416 defines an annular vacuum space 416VS that is in fluid communication with the vacuum region 416VR and that has a generally annular shape. In some embodiments, the inner and outer frames 416FI and 416FO are made of one or more relatively rigid material, such as polycarbonate, polyphenylsulfone, and acrylic, among others. The inner frame 416FI defines a breathing region 416B that, during use, is in fluid communication with a wearer's nose and mouth (not shown). The inner frame 416FI also defines a pressure inlet 416PI that is fluidly isolated from the surrounding vacuum space 416VS and carries pressurized gas that pressurizes the breathing region 416B.

The PDS 404 includes an inner seal 404SI and an outer seal 404SO that are each designed and configured to create as gas-tight a seal as possible with a wearer's face (not shown) when the VP ventilation mask 400 is properly engaged with that face. Each of the inner and outer seals 404SI and 404SO is made of a relatively flexible material, such as, but not limited to silicone rubber and thermoplastic elastomer, among others, to assist with effecting the above-mentioned gas-tight seal between the PDS 404 and the wearer's face. The inner and outer seals 404SI and 404SO form an interseal region 404IR that is in fluid communication with the vacuum region 416VR of the body 416 and the vacuum space 416VS. In this example, the PDS 404 is formed separately from the body 416 and secured thereto at a gas-tight joint 420, which may consist of any suitable type. In other embodiments, the PCS 404 may be formed integrally with the body 416, for example using an overmolding technique, among others.

In this embodiment, the gas-handling component 400G includes the vacuum ejector 408 and various gas passageways relating thereto and for delivering to and exhausting gas from the breathing region 416B via the pressure inlet 416PI. In particular, the gas-handling component 400G includes a pressure passageway 424 and a vacuum passageway 428. The pressure passageway 424 has a pressurized-gas inlet 424I that, when the VP ventilation mask 400 is in use, receives pressurized gas from a pressurized-gas source (not shown, but see, e.g., pressurized-gas sources 212 and 312 of FIGS. 2 and 3, respectively). The vacuum passageway 428 is in fluid communication with the PDS 404 via the vacuum space 416VS and the vacuum region 416VR. In the embodiment shown, the vacuum passageway 428 is generally cylindrical in shape to match the generally annular shape of the vacuum space 416VS of the face-engaging component 400F. In other embodiments, the vacuum passageway 428 and/or vacuum space 416VS may have other shapes and/or be executed with multiple individual passageways that collectively function as a single passageway.

The vacuum ejector 408 includes a motive-flow, or venturi, nozzle 408V that, during operation, receives a primary pressurized flow from the pressure passageway 424 so as to create a low-pressure zone, which is in fluid communication with the vacuum passageway 428. The resulting low gas pressure within the low-pressure zone of the venturi nozzle 408V draws a vacuum within the vacuum passageway 428 and, correspondingly, within the interseal region 404IR of the PDS 404. The vacuum ejector 408 also includes a diffuser 408D that, when the VP ventilation mask 400 is operating, mixes the flows from the pressure passageway 424 and the vacuum passageway 428, decelerates the combined flow to recover static pressure, and exhausts the combined flow stream at pressure slightly above ambient pressure (P₀).

The following examples in the context of the VP ventilation mask 400 of FIGS. 4A through 4C provide the reader with some measure of scale for components of some instantiations of such VP ventilation mask and other VP ventilation masks made in accordance with the present disclosure. In some instantiations, the transverse cross-sectional flow area of the venturi nozzle 408V may be in a range from 8 mm² to 40 mm², inclusive. In other embodiments: the same range may be used for the sum of multiple venturi nozzles; the length of the vacuum ejector 408 from the venturi nozzle 408V to the exit of the diffuser 408D may be in a range from 25 mm to 175 mm; the minimum transverse cross-sectional area, i.e., throat 408T, within the diffuser may be in a range from 20 mm² to 120 mm²; and the diffuser may be a divergent diffuser having a divergence angle from 2° to 20°.

As described above relative to the VP mask system 200 of FIG. 2, a principle of operation of a VP ventilation mask of the present disclosure, including the VP ventilation mask 400 of FIGS. 4A through 4C, is that the vacuum pressure P_{V} in the interseal region, here, interseal region 404IR, is less than both the ventilation pressure P₁ in the breathing region, here breathing region 416B, and the ambient pressure P₀ in the ambient environment, here, ambient environment 432. In order to achieve this pressure relationship, the venturi nozzle 408V needs to be designed in conjunction with the ventilation pressure P₁ and ventilation flow, the vacuum pressure P_{V} and vacuum flow, and the ambient pressure P₀, among other variables. Those skilled in the art will understand how to design a suitable venturi nozzle, such as the venturi nozzle 408V, to create a venturi nozzle that provides the requisite flows to achieve the desired relationship of P_{V}<P₀<P₁.

In the embodiment shown, the pressurized-gas inlet 424I is provided by an inlet fitting 436 that is designed and configured to fluidly connect the VP ventilation mask 400 to a pressurized-gas delivery conduit (not shown), such as a tube. Also in the embodiment shown, the gas exhausted by the diffuser 408D of the vacuum ejector 408 is expelled through an exhaust outlet fitting 440 that is designed and configured to fluidly connect the VP ventilation mask 400 to an exhaust handler (not shown), such as tubing and/or a viral filter, among other things.

As seen in FIGS. 4B and 4C in particular, the embodiment of the VP ventilation mask 400 includes an anti-asphyxiation valve 444 that includes an opening 444O that receives a movable valve member 444M (FIG. 4C) that seals the anti-asphyxiation valve opening when fresh breathing gas is flowing from a gas source and into the VP ventilation mask via the pressurized gas inlet 424I. As known in the art, the anti-asphyxiation valve 444 prevents the wearer from becoming asphyxiated when there is a failure in providing fresh clean pressurized gas to the breathing region 416B, such as when a pressurized-gas source loses power or a component of the pressurized-gas source (not shown) fails. Typically, the anti-asphyxiation valve member 444M comprises a flexible silicone or other polymeric membrane that opens to allow the wearer's breathing to draw fresh air from the ambient environment 432 into the breathing region 416B via the opening 444O when such a failure occurs.

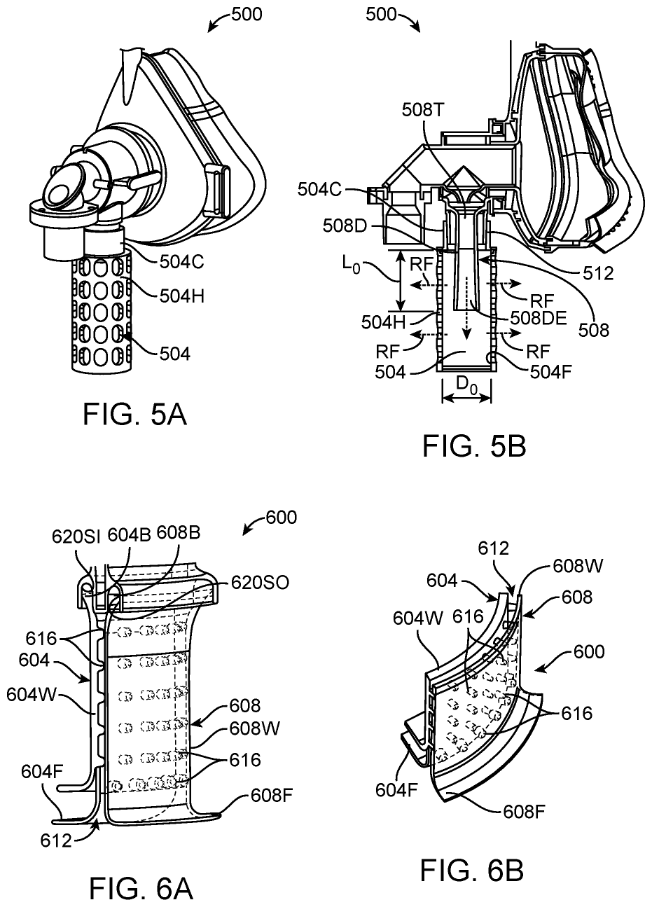

FIGS. 5A and 5B show another example VP ventilation mask 500 made in accordance with the present disclosure that includes an integrated viral-filter assembly 504 that includes a filter membrane 504M contained in an apertured housing 504H. In the embodiment shown, the viral-filter assembly 504 has a cylindrical form factor that provides substantially more total effective filtration area (e.g., two or more times the effective area) than a standard commercial filter of similar size to minimize pressure drop, while maintaining a very compact mask/filter assembly that is unobtrusive to the wearer. In this example, portions of the filter membrane 504M and the housing 504H surround a diffuser 508D (FIG. 5B) of a vacuum ejector 508 that is similar to the vacuum ejector 408 of FIGS. 4A through 4C. In other words, at least a portion of the diffuser 508D extends into a central region 504CR of the viral-filter assembly 504, here by an overlap length, L_{O}. As those skilled in the art will readily appreciate, by locating at least some of the filter membrane 504M above (relative to FIG. 5B) the exit 508DE of the diffuser 508D, the overall size of the VP ventilation mask 500 can be more compact, while also allowing for an increased flow area through the filter membrane 504M.

Conventional viral-filter membranes are typically planar disks. However, the filter membrane 504M of the viral-filter assembly 504 is cylindrical in shape and provides a radial flow arrangement, as illustrated by radial-flow arrows RF. Consequently, it can be formed, for example, by curving a rectangular membrane into a cylinder and sealing the abutting or overlapping ends of the rectangular member together with one another or to another member (not shown), for example, using ultrasonic welding or adhesive bonding, among others. In some embodiments, the filter member 504M may be secured to the housing 504H, or to an inner frame (not shown) within the central region 504CR, or both. In some embodiments, a circular piece of filter membrane may be used to close the end of the cylinder that is opposite the diffuser 508D. It is noted that the viral-filter assembly 504 may have a tubular shape that is not cylindrical in transverse cross-section. For example, the viral-filter assembly 504 may have a rectangular or oval transverse cross-sectional shape, among others. Those skilled in the art will readily appreciate the various ways in which the viral-filter assembly 504 can be constructed.

To accommodate the viral-filter assembly 504, the VP ventilation mask 500 includes a receiver 512 (FIG. 5B) for receiving a corresponding connector 504C on the viral-filter assembly. In this example, the connector 504C and the receiver 512 engage with one another, such as using a straight threaded or tapered interference connection, such as an ISO (International Standards Organization) tapered connection, among others, to effect a fluid seal. In this example, the connector 504C is a male ISO tapered connector, and the receiver 512 is a female ISO tapered connector. However, this can be reversed in other embodiments. Also, fluid-tight seals other than threaded or interference seals may be used in other embodiments as desired. Other aspects and features of the VP ventilation mask 500 of FIGS. 5A and 5B may be the same as or similar to like aspects and features of the VP ventilation mask 400 of FIGS. 4A through 4C. Benefits of a VP ventilation mask having an integrated viral filter, such as the VP ventilation mask 500 of FIGS. 5A and 5B include but are not limited to:
it creates a more compact package for a ventilation mask than existing commercial filters which makes the mask less obtrusive to wearers in operation;
the large filter area decreases flow resistance, thereby improving performance of the vacuum ejector 508;
the large filter area reduced the potential for clogging of the filter membrane 504M; and
when a standard ISO connector is used for the receiver 512, such connector allows for connecting tubing to handle the exhaust from the VP ventilation mask 500 in cases wherein that is desired.

FIGS. 6A and 6B illustrate an example construction that can be used for a PDS 600 of a VP ventilation mask (not shown) of the present disclosure, such as any of the VP ventilation masks 208, 400, and 500 of FIGS. 2, 4A-4C, and 5A-5B, respectively, among others. In this example, the PDS 600 of FIGS. 6A and 6B includes an inner-seal structure 604 and an outer-seal structure 608 that are made of a flexible elastomer, such as silicone rubber, that allows each of their face-engaging flanges 604F and 608F to readily conform to the shape of a wearer's face (not shown) to effect an effective gas-tight seal between the inner- and outer-seal structures and the face.

Each of the inner- and outer-seal structure 604 and 608 has a corresponding wall 604W and 608W, and these wall define an interseal region 612 therebetween that contains the vacuum pressure P_{V} (see, e.g., FIG. 2 and corresponding description above) during use of the VP ventilation mask with which the PDS 600 is used. The PDS 600 also includes anti-collapse features 616, here, cylindrical columns (only a few labeled to avoid clutter), that prevent the vacuum pressure P_{V} from drawing the walls 604W and 608W into contact with one another so as to close the interseal region 612 and preventing the vacuum pressure from reaching the wearer's face during use. It is important to maintain an open flow path for the vacuum purge flow in the interseal region 612 to remove any contaminants that may leak between the face-engaging flanges 604F and 608F of the inner- and outer-seal structures 604 and 608 during use of the VP ventilation mask as described above.

The anti-collapse features 616 may be provided in any suitable manner, such as molded into one, the other, or both of the walls 604W and 608W or provided in an insert (not shown) that is a separate structure from the inner- and outer seal structures 604 and 608. The size(s), number, location, and spacing(s) of the anti-collapse features 616 may be determined as a function of, for example, the magnitude of the vacuum pressure P_{V}, and the flexibility of the walls 604W and 608W, among other variables. As seen in FIG. 6A, each of the inner- and outer-seal structures 604 and 608 includes a corresponding seal-bead 604B and 608B that is engaged, for example, by press fit, in a corresponded inner-seal seat 620SI (FIG. 6A) and outer-seal seat 620SO of a body 620 of a VP ventilation mask so as to form a gas-tight seal.

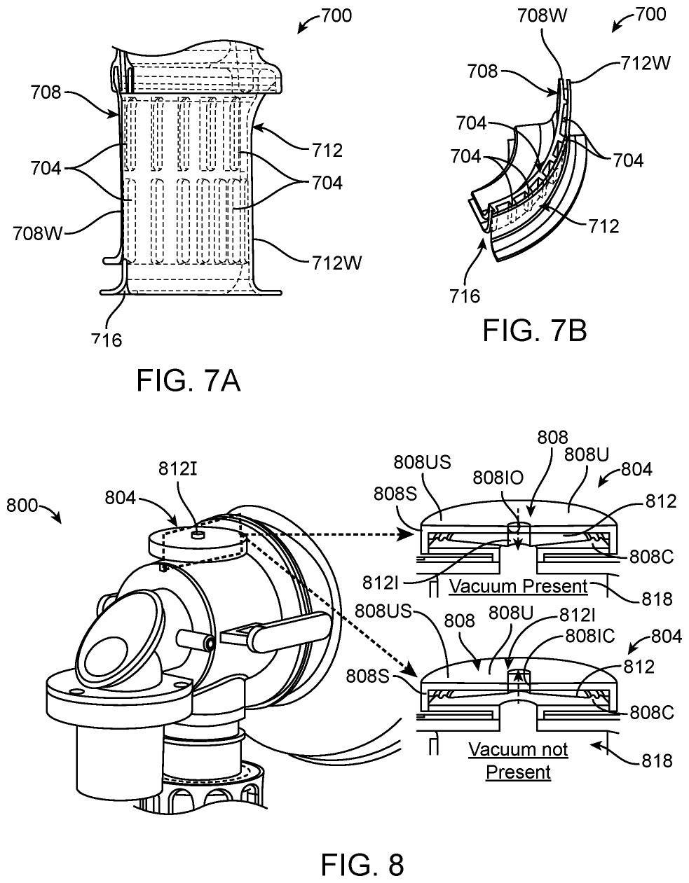

FIGS. 7A and 7B show another example construction that can be used for a PDS 700 of a VP ventilation mask of the present disclosure, such as any of the VP ventilation masks 208, 400, and 500 of FIGS. 2, 4A-4C, and 5A-5B, respectively, among others. In the example of FIGS. 7A and 7B the cylindrical column style anti-collapse features 616 of FIGS. 6A and 6B are replaced by elongated-wall-type anti-collapse features 704 (only a few labeled to avoid clutter). As with the anti-collapse features 616 of FIGS. 6A and 6B, the anti-collapse features 704 of FIGS. 7A and 7B may be provided in any suitable manner, such as molded into one, the other, or both of the walls 708W and 712W of the inner- and outer-seal structures 708 and 712 or provided in an insert (not shown) that is a separate structure from the inner- and outer seal structures. The, size(s), number, location, and spacing(s) of the anti-collapse features 704 may be determined as a function of, for example, the magnitude of the vacuum pressure P_{V} (see, e.g., FIG. 2) present within the interseal region 716 of the PDS 700 during use, and the flexibility of the walls 708W and 712W, among other variables. Other features and aspects of the PDS 700 of FIGS. 7A and 7B may be the same as or similar to the like features and aspects of the PDS 600 of FIGS. 6A and 6B.

FIG. 8 illustrates an example VP ventilation mask 800 that includes a vacuum indicator 804 integrated into a gas-handling component 808 of the VP ventilation mask. In this example, the vacuum indicator 804 includes a housing 808 and a pressure diaphragm 812 that is tuned to respond to the vacuum pressure P_{V} (see, e.g., FIG. 2 and corresponding description) that is present in the interseal region of a PDS of the VP ventilation mask 800 (interseal region not shown, but see, e.g., interseal region 220IR of the PDS 220 of FIG. 2). The upper wall 808U of the housing 808 includes an indicator opening 808IO, and the pressure diaphragm 812 has an attached visual indicator 812I that is engaged within the indicator opening and moves with the pressure diaphragm.

The pressure diaphragm 812 is secured to the sidewall 808S of the housing in a gas-sealed manner so as to define a pressure chamber 808C below (relative to FIG. 8) the pressure diaphragm. The pressure chamber 808C is in fluid communication with a vacuum passageway 816 of the gas-handling component that in subjected to the vacuum-pressure P_{V} one side, and the ambient pressure P₀ on the other side during proper operation of the VP ventilation mask 800. The vacuum indicator 804 is configured so that A) when the vacuum pressure P_{V} is not present within the vacuum passageway 818 (and also not in the PDS), the visual indicator 812I extends beyond the upper surface 808US (relative to FIG. 8) of the upper wall 808U of the housing 808 and B) when the vacuum pressure P_{V} is present within the vacuum passageway (and also in the PDS), the visual indicator does not extend beyond the upper surface of the upper wall of the housing. The visual indicator 812I may be provided with one or more colors, such as red, to make it visually stand out relative to the upper wall 808U of the housing 808. In this embodiment, when a wearer is wearing the VP ventilation mask 800 and the visual indicator 812I extending above the upper surface 808US of the housing 808, this is a signal to an attendant (e.g., healthcare worker) of the wearer that a proper purging vacuum does not exist between the PDS and the wearer's face. The attendant may then take the appropriate action, such as reposition the VP ventilation mask 800 on the wearer's face, check gas-conduit connections, replace the viral filter on the exhaust, etc., to fix the issue. It is noted that features and aspects of the VP ventilation mask 800 may be the same as or similar to any of the VP ventilation masked disclosed elsewhere herein, such as VP ventilation masks 208, 400, and 500 of FIGS. 2, 4A-4C, and 5A-5B, respectively.

### Vacuum Ejector Performance Testing

The performance of a vacuum ejector of a VP ventilation mask of the present disclosure, such as any of VP ventilation masks 208, 400, 500, and 800 of FIGS. 2, 4A-4C, 5A-5B, and 8, sets the limits of mask leak rate which can be effectively captured and filtered by the VP ventilation mask. The vacuum ejector 508 shown in FIGS. 5A and 5B, as integrated into the VP ventilation mask 500, was tested using a laboratory test bench setup. The integrated viral-filter assembly 504 shown in FIGS. 5A and 5B was included on the exhaust end of the vacuum ejector 508. The length from the nozzle throat 508T of the diffuser 508 to the diffuser exit 508DE was 59 mm. The length from the nozzle throat 508T to the distal end 504DE of the viral-filter assembly 504 was 90 mm. The outer diameter, D_{O}, of the viral-filter assembly 504, was 33 mm. The vacuum ejector 508 was connected to a V60 ventilator available from Koninklijke Philips N. V., which provided fixed pressures simulating the internal pressure of the VP ventilation mask. The motive flow rate and gauge pressure were measured and recorded. The vacuum-purge-flow portion of the vacuum ejector 508 was connected to an adjustable leak-flow valve to facilitate a variable flow resistance. This vacuum flow gauge pressure and flow rate were measured just upstream of the vacuum ejector 508. Performance curves of the vacuum pressure as a function of leak rate for the vacuum ejector 508 were obtained by varying the position of the leak-flow valve. Resulting data were recorded for motive pressures of 4 cmH₂O, 12 cmH₂O, and 20 cmH₂O supplied to the vacuum ejector 508, simulating mask CPAP pressures.

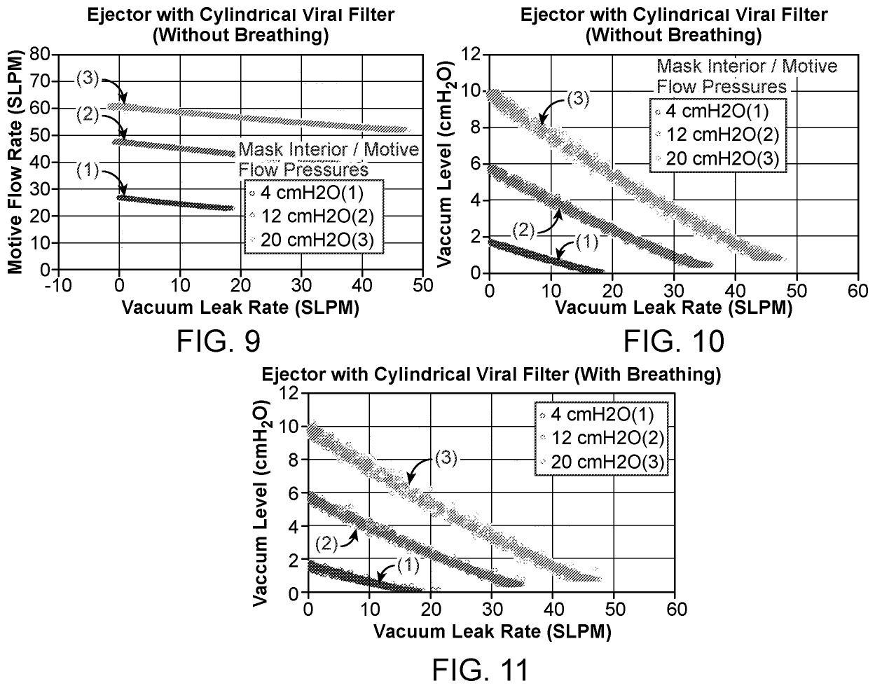

FIG. 9 shows the motive flow rate of the vacuum ejector 508 as a function of vacuum purge leak rate for three different motive flow pressures. The motive flow rate is equivalent to the primary mask exhaust flow rate, while the motive flow pressure is equivalent to a mask CPAP or BiPAP pressure. The data show that the ejector motive flow rate is similar to the flow rate through existing NIV circuits operating at the same pressures. Variation in the leak rate has only a minor impact on the motive flow rate of the vacuum ejector 508 for a fixed supply pressure.

FIG. 10 shows the vacuum pressure of the vacuum ejector 508 as a function of vacuum leak rate for three motive flow pressures. The vacuum pressure is equivalent to the purge pressure in the interseal region of the PDS of the VP ventilation mask. The performance of the vacuum ejector 508 increased as the motive flow pressure increased. This is important, because it is expected that leakage of the VP ventilation mask will increase as the pressure within the breathing region increases. Therefore the vacuum ejector will need to be able to create a high flow rate in the vacuum-purged interseal region. The vacuum ejector 508 tested was able to maintain a vacuum pressure in the vacuum-purge interseal region for flow rates above 15 slpm (standard liters per minute), 30 slpm, and 40 slpm for motive flow pressures of 4 cmH₂O, 12 cmH₂O, and 20 cmH₂O respectively. These flow rates are greater than 50% of the nominal motive flow rate of the VP ventilation mask in all cases (shown in FIG. 9). Thus the vacuum ejector 508 was able to maintain a vacuum pressure in the vacuum-purged interseal region with leak rates of >50% of the mask motive flow rate.

Performance testing of the vacuum ejector 508 was repeated with a breathing machine connected to the motive flow line of the vacuum ejector. The breathing machine simulated the inhalation and exhalation of a person wearing a VP ventilation mask containing the vacuum ejector 508. The performance of the vacuum ejector 508 with this simulated breathing is shown in FIG. 11. The performance is unchanged when compared to the no breathing case shown in FIG. 10. This is expected due to the flow source provided by the V60 ventilator operating in a CPAP mode. The V60 ventilator compensated for breathing and maintained a constant pressure within the breathing region of the ventilation mask. With a constant pressure within the breathing region of the VP ventilation mask, the motive flow pressure did not vary. Therefore performance of the vacuum ejector 508 was not affected by breathing.

### PP Ventilation Mask

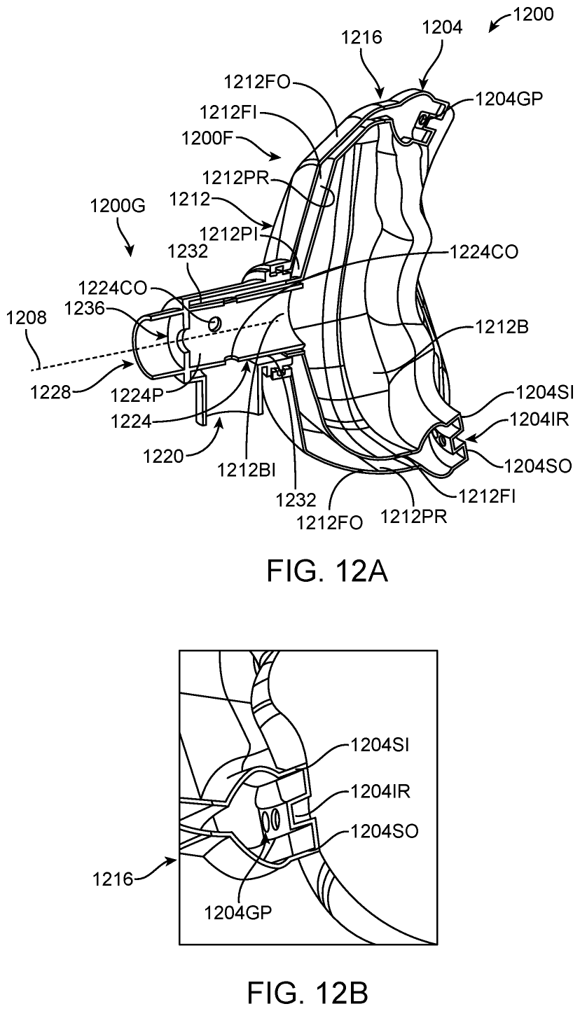

FIGS. 12A and 12B illustrates an example PP ventilation mask 1200 that incorporates features and aspects of the present disclosure, including a PDS 1204. At a high level, this instantiation of the PP ventilation mask 1200 may be considered to have face-engaging component 1200F and a gas-handling component 1200G, which in this example is coupled to the face-engaging component so as to be pivotable relative to the face-engaging component about a pivot axis 1208.

In this embodiment, the face-engaging component 1200F includes the PDS 1204, which is secured to a body 1212 that includes an inner frame 1212FI and an outer frame 1212FO that are spaced from one another so as to define a PDS-pressurizing region 1212PR therebetween. In this example, the body 1212 defines a PDS-pressurizing inlet 1212PI that is in fluid communication with PDS-pressurizing region 1212PR and that has a generally annular shape. In some embodiments, the inner and outer frames 1212FI and 1212FO are made of one or more relatively rigid material, such as polycarbonate, polyphenylsulfone, and acrylic, among others. The inner frame 1212FI defines a breathing region 1212B that, during use, is in fluid communication with a wearer's nose and mouth (not shown). The inner frame 1212FI also defines a breathing-pressure inlet 1212BI that is fluidly isolated from the surrounding PDS-pressurizing inlet 1212PI and carries-reduced-pressure gas that pressurizes the breathing region 1212B as described below in detail.

The PDS 1204 includes an inner seal 1204SI and an outer seal 1204SO that are each designed and configured to create as gas-tight a seal as possible with a wearer's face (not shown) when the VP ventilation mask 1200 is properly engaged with that face. Each of the inner and outer seals 1204SI and 1204SO is made of a relatively flexible material, such as, but not limited to silicone rubber, thermoplastic elastomer, among others, to assist with effecting the above-mentioned gas-tight seal between the PDS 1204 and the wearer's face. The inner and outer seals 1204SI and 1204SO form an interseal region 1204IR that is in fluid communication with the PDS-pressurizing region 1212PR of the body 1212 and the PDS-pressurizing inlet 1212PI via a PDS-pressurizing region 1204P within the PDS 1204. In this example, the PDS 1204 is formed separately from the body 1212 and secured thereto at a gas-tight joint 1216, which may of any suitable type. In other embodiments, the PCS 1204 may be formed integrally with the body 1212, for example using an overmolding technique, among others.

In this example, the interseal region 1204IR is in fluid communication with the PDS-pressurizing region 1204P of the PDS 1204 and, therefore, also the PDS-pressurizing region 1212PR of the body 1212 and the PDS-pressurizing inlet 1212PI, via a gas-permeable structure 1204GP, here a perforated barrier. In this embodiment, the relatively high seal pressure P_{P} (see, e.g., FIG. 3 and accompanying description) inside the PDS-pressurizing region 1204P of the PDS 1204 assists in maintaining the shapes of the inner and outer seals 1204SI and 1204SO during operation of the PP ventilation mask 1200, and the gas-permeable structure 1204GP also assists in maintaining the shapes of the inner and outer seals during operation. In other embodiments, the gas-permeable structure 1204GP may have a different structure, such as, an open-cell foam or an electrospun permeable structure, among others. In some embodiments, a gas-permeable structure, such as the gas-permeable structure 1204GP, need not be provided such that the interseal region 1204IR is in direct fluid communication with the PDS-pressurizing region 1204P of the PDS 1204.

In this embodiment, the gas-handling component 1200G includes a pressurized-gas inlet 1220, a passive pressure reducer 1224, and a gas outlet 1228. During use, the pressurized-gas inlet connects to a pressurized-gas source (not shown, but that may be the same as or similar to the pressurized gas source 312 of FIG. 3) that provides pressurized gas nominally at the relatively higher seal pressure P_{P}. The pressurized-gas inlet 1220 provides its flow directly to a PDS-pressurizing passageway 1228 that in turn provides the seal pressure P_{P} to the interseal region 1204IR via the PDS-pressurizing inlet 1212PI, the PDS-pressurizing region 1212PR, the PDS-pressurizing region 1204P, and the gas-permeable structure 1204GP.

The passive pressure reducer 1224 defines a breathing-gas passageway 1224P that is in direct fluid communication with the breathing region 1212B of the face-engaging body 1212 and includes a plurality of constricting orifices 1224CO (only some shown and labeled) that fluidly connect the breathing-gas passageway with the PDS-pressurizing passageway 1232. As noted above, for example, relative to FIG. 3, during operation of the PP ventilation mask 1200 the PDS-pressurizing passageway 1232 is at a pressure P_{P}. The constricting orifices 1224CO constrict the flow of the pressurized gas from the PDS-pressurizing passageway 1232 to the breathing-gas passageway 1224P, thereby causing the breathing pressure P₁ (see, e.g., FIG. 3) in the breathing-gas passageway to be lower than the pressure P_{P} (see, e.g., FIG. 3) in the PDS-pressurizing passageway. The relationship among the PDS-pressurizing pressure P_{P}, the breathing pressure P₁, and the ambient pressure P₀ surrounding the PP ventilation mask 1200 during use is described above in detail relative to FIG. 3.

To summarize that description of FIG. 3, pressurized gas nominally at the PDS-pressurizing pressure P_{P} that is higher than the breathing pressure P1 is provided to the PP ventilation mask 1200 via the pressurized-gas inlet 1220, and the passive pressure reducer 1224 lowers PDS-pressurizing pressure P_{P} to the desired breathing pressure P₁. The gas outlet 1228 can be fluidly connected, for example, to a gas conduit (not shown) or a viral filter (not shown), or both, or other exhaust-gas handler. In this example, the gas-handling component 1200G includes a flow restrictor 1236, here, a constricting orifice, between the breathing-gas passageway 1224P and the gas outlet 1228 to reduce the breathing-gas pressure P₁ (see, e.g., FIG. 3) inside the breathing-gas passageway to ambient pressure P₀ or to a pressure between P₁ and P₀ as may be needed to accommodate and downstream component(s) (not shown). In some embodiments, the flow restrictor 1236 may be of a type difference from the single constrictive orifice shown. In some embodiments, the flow restrictor 1236 may be eliminated and the pressure differential between P₁ and P₀ handled outside of the PP ventilation mask 1200, such as in a viral filter system (not shown).

Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A ventilation mask for ventilating a wearer of the ventilation mask via at least one of a nose and a mouth of the wearer when the ventilation mask is surrounded by an ambient environment having an ambient pressure, P₀, the ventilation mask comprising:
a breathing region designed and configured to receive the at least one of the nose and the mouth and contains breathing gases at a breathing pressure, P₁, when the ventilation mask is in use, wherein the breathing region has a periphery;
an inner seal that defines the periphery of the breathing region and is designed and configured to engage the wearer during use of the ventilation mask so as to create a first gas seal between the wearer and the ventilation mask so as to seal the breathing region;
an outer seal spaced from the inner seal so as to define an interseal region between the inner seal and the outer seal, the outer seal designed and configured to engage the wearer during use of the ventilation mask so as to create a second gas seal between the wearer and the ventilation mask so as to seal the interseal region;
a gas-handling component that has either:
a vacuum-purge-pressure configuration by which, during use, the gas-handling component receives a fresh breathing gas from a gas source at the breathing pressure P₁ and includes a vacuum generator that is in fluid communication with, separately, each of the breathing region and the interseal region, wherein the vacuum generator is designed and configured to, during use, exhaust the breathing gases from the breathing region and use the breathing gases at the breathing pressure P₁ to create a vacuum purge pressure, P_{V}, in the interseal region, wherein P_{V}<P₀<P₁; or
a positive-purge-pressure configuration by which, during use, the gas-handling component receives the fresh breathing gas from the gas source at a positive purge pressure, P_{P}, that is higher than the breathing pressure P₁, provide the fresh breathing gas to the interseal region at the positive purge pressure P_{P}, and provide the fresh breathing gas received at the positive purge pressure P_{P} to the breathing region at the breathing pressure P₁, wherein P_{P}>P₁>P₀.

2. The ventilation mask of claim 1, wherein the gas-handling component has the vacuum-purge configuration.

3. The ventilation mask of claim 2, wherein the vacuum generator includes a vacuum ejector having a venturing nozzle that, during use, receives the breathing gases from the breathing region at the breathing pressure P₁ and generates therefrom the vacuum purge pressure P_{V} in the interseal region.

4. The ventilation mask of claim 3, wherein the vacuum ejector includes a diffuser located fluidly downstream from the venturi nozzle.

5. The ventilation mask of claim 3, further comprising a viral filter located fluidly downstream from the venturi nozzle.

6. The ventilation mask of claim 5, wherein the viral filter is removably secured to the vacuum ejector.

7. The ventilation mask of claim 5, wherein the vacuum ejector includes a diffuser having a longitudinal central axis and located fluidly downstream from the venturi nozzle, wherein the viral filter defines a central region into which the diffuser extends so that the viral filter and the diffuser overlap one another in a direction along the longitudinal central axis.

8. The ventilation mask of claim 2, further comprising a vacuum indicator that, during use of the ventilation mask, indicates presence of the vacuum purge pressure P_{V} in the interseal region.

9. The ventilation mask of claim 8, wherein the vacuum indicator includes a pressure diaphragm in fluid communication with the interseal region.

10. The ventilation mask of claim 8, wherein the vacuum indicator is part of the gas-handling component.

11. The ventilation mask of claim 8, wherein the vacuum indicator includes a visual indicator.

12. The ventilation mask of claim 2, further comprising a face-engaging component coupled to the gas-handling component, wherein the face-engaging component includes:
an inner frame that defines, in conjunction with the inner seal, the breathing region; and
an outer frame spaced from the inner frame so as to define a vacuum pathway between the interseal region and the gas-handling component, wherein, during use, the vacuum pathway is at the vacuum purge pressure P_{V}.

13. The ventilation mask of claim 12, wherein the gas-handling component is pivotably coupled to the face-engaging component.

14. The ventilation mask of claim 12, wherein the gas-handling component includes a central pressure passageway in fluid communication with the breathing region and an annular vacuum region surrounding the central pressure passageway and in fluid communication with the interseal region.

15. The ventilation mask of claim 14, wherein the gas-handling component is pivotably coupled to the face-engaging component.

16. The ventilation mask of claim 12, wherein the inner and outer seals include corresponding respective flexible walls that are spaced from one another to form a vacuum space, and the ventilation mask further includes anti-collapse features in the vacuum space that are designed and configured to keep the vacuum space from collapsing during use of the ventilation mask.

17. The ventilation mask of claim 16, wherein the anti-collapse features comprise cylindrical members.

18. The ventilation mask of claim 16, wherein the anti-collapse features comprise elongate walls.

19. The ventilation mask of claim 12, wherein the wearer has a face containing the nose and the mouth, and the inner seal is designed and configured to seal with portions of the face laterally surrounding the nose and the mouth.

20. The ventilation mask of claim 1, wherein the gas-handling component has the positive-purge-pressure configuration.

21. The ventilation mask of claim 20, wherein the gas-handling component includes:
an inlet that receives the fresh breathing gas at the positive purge pressure P_{P} during use; and
a pressure reducer located fluidly between the inlet and the breathing region, wherein the pressure reducer is designed and configured to reduce the positive purge pressure P_{P} to the breathing pressure P₁ during use.

22. The ventilation mask of claim 21, wherein the pressure reducer comprises a flow restrictor having at least one flow-restriction aperture.

23. The ventilation mask of claim 21, wherein the gas-handling component further includes an exhaust outlet in fluid communication with the breathing region so as to be at the breathing pressure P₁ during use.

24. The ventilation mask of claim 20, further comprising a pressure reducer in fluid communication with the breathing region and designed and configured so as to, during use, reduce the breathing pressure P₁ of the breathing gases before the ventilation mask exhausts the breathing gases.

25. The ventilation mask of claim 20, further comprising a face-engaging component coupled to the gas-handling component, wherein the face-engaging component includes:
an inner frame that defines, in conjunction with the inner seal, the breathing region; and
an outer frame spaced from the inner frame so as to define a positive pressure purge pathway between the interseal region and the gas-handling component, wherein, during use, the positive pressure purge pathway is at the positive purge pressure P_{P}.

26. The ventilation mask of claim 25, wherein the gas-handling component is pivotably attached to the face-engaging component.

27. The ventilation mask of claim 26, wherein the gas-handling component includes a central pressure passageway in fluid communication with the breathing region and an annular pressure region surrounding the central pressure passageway and in fluid communication with the interseal region.

28. The ventilation mask of claim 27, wherein the central pressure passageway has an exhaust outlet for exhausting the breathing gases from the breathing region.

29. The ventilation mask of claim 27, wherein the gas-handling component is pivotably attached to the face-engaging component.

30. The ventilation mask of claim 25, further comprising a gas-permeable structure extending between the inner and outer seals.

31. The ventilation mask of claim 25, wherein the wearer has a face containing the nose and the mouth, and the inner seal is designed and configured to seal with the face around the nose and the mouth.

32. A method of ventilating, at a breathing pressure, P₁, higher than an ambient pressure, P₀, a mask wearer having a nose and a mouth via a ventilation mask having a breathing region containing at least one of the nose and the mouth and that has a purged dual seal engaged with the mask wearer and that seals the breathing region, the method comprising either:
providing a vacuum purge pressure, P_{V}, to the purged dual seal so that P_{V}<P₀<P₁, wherein the providing of the vacuum purge pressure P_{V} includes:
receiving fresh breathing gas at the breathing pressure P₁; and
generating the vacuum purge pressure P_{V} from breathing gases exhausted from the breathing region; or
providing a positive purge pressure, P_{P}, to the purged dual seal so that P_{P}>P₁>P₀, wherein the providing of the pressure purge pressure P_{P} includes:
receiving a fresh breathing gas at a positive purge pressure P_{P};
providing the received fresh breathing gas to the purged dual seal at the positive purge pressure P_{P}; and
providing the received fresh breathing gas to the breathing region at the breathing pressure P₁ by reducing pressure of the received fresh breathing gas from the positive purge pressure P_{P} to the breathing pressure P₁.

33. The method of claim 32, comprising the providing of the vacuum purge pressure P_{V} to the purged dual seal so that P_{V}<P₀<P₁.

34. The method of claim 33, wherein generating the vacuum purge pressure P_{V} includes using a vacuum ejector.

35. The method of claim 34, wherein generating the vacuum purge pressure P_{V} includes using a venturi nozzle.

36. The method of claim 33, further comprising filtering both gases drawn out of the purged dual seal and the breathing gases used to generate the vacuum purge pressure P_{V}.

37. The method of claim 36, further comprising diffusing both the gases drawn out of the purged dual seal and the breathing gases used to generate the vacuum purge pressure P_{V}.

38. The method of claim 33, further comprising providing an indication that the vacuum purge pressure P_{V} is present in the purged dual seal.

39. The method of claim 32, comprising the providing of the positive purge pressure P_{P} to the purged dual seal so that P_{P}>P₁>P₀.

40. The method of claim 39, wherein the reducing the pressure of the received fresh breathing gas from the positive purge pressure P_{P} to the breathing pressure P₁ includes passing the fresh breathing gas through a pressure reducer having a flow restrictor.

41. The method of claim 40, wherein the flow restrictor has at least one flow-restriction aperture.

42. The method of claim 39, further comprising exhausting breathing gases from the breathing region.

43. The method of claim 42, wherein exhausting the breathing gases includes passing the breathing gases through a pressure reducer so as to reduce the pressure of the breathing gases from the breathing pressure P₁ to a predetermined pressure.

44. The method of claim 42, further including filtering the exhausted breathing gases.
